(19) United States
(12) Patent Application Publication
(10) Pub. No.: US 2010/0186152 A1
(43) Pub. Date: Jul. 29, 2010

(54) Light Attenuating Shield for a Motorcycle Helmet Visor

(76) Inventor: Phillip Freeman, Fortuna, CA (US)

Correspondence Address: Russ Weinzimmer, 614 Nashua Street, Suite 53, Milford, NH, 03055, US

(21) Appl. No.: 12/359,292

(22) Filed: Jan. 24, 2009

(51) Int. Cl. A42B 1/24; A61F 9/00; A42B 3/22

(52) U.S. Cl. 2/422; 2/10; 2/424

(57) ABSTRACT

A lightweight, flexible, light-attenuating sun and glare shield is disclosed that is attachable via static electricity to the inside of a motorcycle helmet visor. No modification of the motorcycle helmet or visor is required for attachment of the shield, and the shield need not be specially configured for a specific model of helmet, so long as the shape and size of the shield is generally compatible with the visor. The shield is easily removed from the visor and rolled up for convenient transportation and storage. In preferred embodiments, the light attenuating shield is polarized and/or includes graduated tinting. Some embodiments include a storage canister for easy transportation and storage of the shield, and certain embodiments include a plurality of shields, which can be disposable and/or can include shields with different light attenuating properties.

### FIELD OF THE INVENTION

The invention generally relates to motorcycle helmets, and more specifically to accessory attachments for motorcycle helmets.

### BACKGROUND OF THE INVENTION

For both safety and comfort, motorcycle riders generally wear helmets while riding. In addition to protecting a rider's head in case of an accident, motorcycle helmets are frequently also equipped with a transparent visor so as to deflect wind, debris, and other contaminants away from a rider's face, especially when the motorcycle is traveling at high speeds. Often, the visor is detachable from the helmet, so as to allow a rider's face to be unobstructed when traveling at lower speeds.

Sun and headlight glare can cause significant discomfort for a motorcycle rider, and can also create unsafe riding conditions. Tinted motorcycle helmet visors are available that can help to reduce the effects of sun and headlight glare. However, it may not be desirable to use a tinted helmet visor under all conditions, such as on a cloudy day or at night on a sparsely traveled road. This can require a motorcycle rider to carry and exchange multiple visors, both tinted and non-tinted, which can be cumbersome and inconvenient.

Similarly, exchangeable motorcycle helmet visor shields are available that can be worn either in front of or behind a visor, some of which are tinted and/or photosensitive. However, these visor shields must be configured for attachment to a specific model of motorcycle helmet, and/or require special adaptations to be installed on the visor, such as hook-and-loop attachment strips. Also, it can be inconvenient and cumbersome to carry and store a visor shield when it is not in use.

### SUMMARY OF THE INVENTION

A lightweight, flexible, light attenuating shield is claimed that is attachable to the inside of a motorcycle helmet visor by static electricity so as to provide protection from sun and headlight glare. No modification of the motorcycle helmet or visor is required for attachment of the shield, and the shield need not be specially configured for a specific model of helmet, so long as the shape of the shield is generally compatible with the shape of the visor.

The claimed shield is easily removed from the visor and rolled up for convenient transportation and storage. In preferred embodiments, the light attenuating shield is polarized and/or includes graduated tinting. In certain embodiments, the light attenuating shields are disposable. Some embodiments include a storage canister for easy transportation and storage of the shield, and various embodiments include a plurality of light attenuating shields with different light attenuating properties.

The invention is a light attenuating shield attachable to a motorcycle helmet visor. The light attenuating shield includes a flexible sheet of polymeric film, the flexible sheet being shaped so as to conform to a portion of an inner surface of a motorcycle visor, the flexible sheet being able to attenuate light passing through the flexible sheet, and the flexible sheet being removably attachable to the portion of the inner surface of the motorcycle visor by static electric attraction.

In preferred embodiments, the flexible sheet is tinted. In other preferred embodiments, n the flexible sheet attenuates light in a non-uniform manner across the sheet. In still other preferred embodiments, the flexible sheet polarizes light passing therethrough. And in some of these embodiments light passing through the sheet is polarized so as to provide maximum attenuation of reflected glare.

In various preferred embodiments, the light attenuating shield further includes at least one additional flexible sheet of polymeric film. In some of these embodiments, a light attenuation characteristic of a first flexible sheet of polymeric film is different from a light attenuation characteristic of a second flexible sheet of polymeric film. And in other of these embodiments at least two flexible sheets of polymeric film can be overlaid and simultaneously attached to the inside of the helmet visor.

In certain preferred embodiments the flexible sheet is disposable.

In preferred embodiments the flexible sheet is sufficiently flexible to enable transportation and storage of the flexible sheet in a rolled configuration. Some of these preferred embodiments further include a storage canister within which the flexible sheet can be stored when the flexible sheet is in a rolled configuration, while other of these preferred embodiments further include a rod for rolling the flexible sheet into a rolled configuration. And in some of these preferred embodiments the rod includes an elongated slot into which an end of the flexible sheet can be inserted so as to facilitate rolling of the flexible sheet.

### BRIEF DESCRIPTION OF THE DRAWINGS

The invention will be more fully understood by reference to the detailed description, in conjunction with the following figures, wherein:

FIG. 1 is a perspective view of a motorcycle rider wearing a helmet with a light attenuating shield attached to the helmet visor, showing light shining on the visor and being blocked by a tinted shield;

FIG. 2 is a perspective view of the embodiment of FIG. 1, showing light reflected from a puddle in a road onto the motorcycle helmet visor and being blocked by a polarizing shield;

FIG. 3 is a perspective close-up view of the embodiment of FIG. 1, showing a light attenuating shield attached to a removable motorcycle helmet visor, except for one corner of the shield that is pealed away from the visor;

FIG. 4 is a perspective view of the preferred embodiment that includes a light attenuating shield, a rod for rolling the shield, and a storage canister;

FIG. 5 is a perspective view of the embodiment of FIG. 4, showing the light attenuating shield rolled around the rod and inserted into a storage canister; and

FIG. 6 is a perspective view of a box containing a plurality of light attenuating shields contained in storage canisters.

### DETAILED DESCRIPTION OF PREFERRED EMBODIMENTS

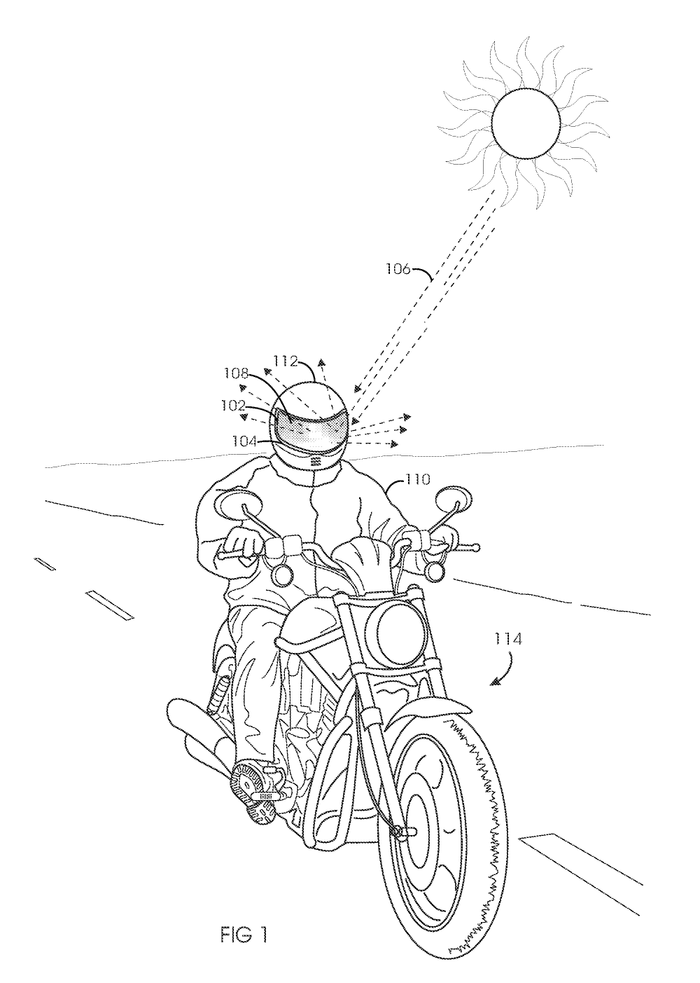

With reference to FIG. 1, the present invention is a light attenuating shield 102 attachable to the inside of a motorcycle helmet visor 104 for attenuating light 106 passing through the visor 104. The shield 102 is made of a flexible sheet 108 of polymeric film. In the embodiment of FIG. 1, the flexible sheet 108 is tinted so as to attenuate light 106 in a non-uniform manner across the flexible sheet 108, thereby enhance the vision of a motorcycle rider 110 riding a motorcycle 114 while wearing a helmet 112.

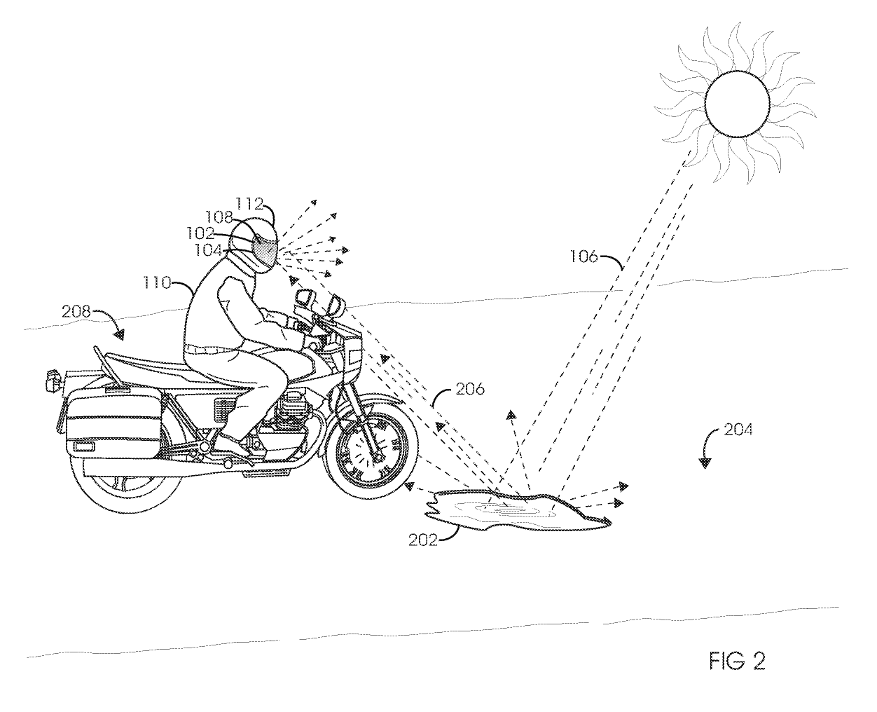

FIG. 2 is a perspective view of the preferred embodiment of FIG. 1, showing light 106 reflected onto the motorcycle helmet visor 104 from a puddle 202 in a road 204. In the embodiment of FIG. 2, the flexible sheet 108 is polarized so as to block reflected glare 206 from the puddle 202 while providing uninterrupted vision for the rider 110 on the motorcycle 114.

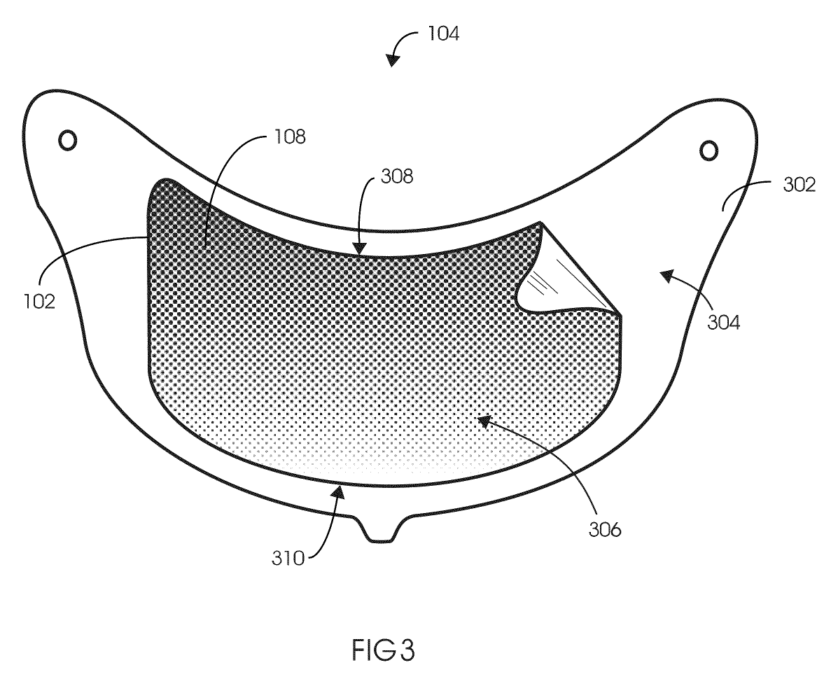

FIG. 3 is a perspective view of the motorcycle helmet visor 104 of FIG. 1 and FIG. 2 showing the light attenuating shield 102 applied to the visor 104 but pealed back from one corner 302 of the visor 104. The flexible sheet 108 of polymeric film is shaped so as to be generally compatible with the inner region 304 of the motorcycle helmet visor 104, and is removably attachable to the inner region 304 of the motorcycle visor 104 by static electric attraction.

In the embodiment of FIG. 3, the flexible sheet 108 is formulated to include graduated tinting 306 for efficient attenuation of sunlight 106 while providing less attenuation of the view directly in front of the motorcycle 114. In other embodiments, the shield 102 is uniformly tinted, or is more heavily tinted on one side so as to provide optimal attenuation of glare from oncoming headlights. No modification of the motorcycle helmet 112 or visor 104 is required for attachment of the shield 102, and the shield 102 need not be specially configured for a specific model of helmet 112, so long as the shape of the shield 102 is generally compatible with the shape of the visor 104.

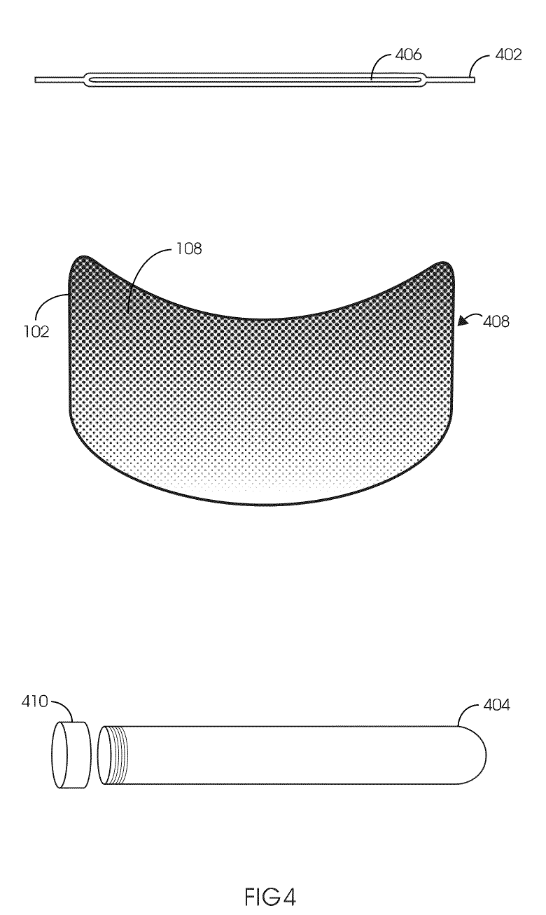

FIG. 4 is a perspective view of an embodiment similar to the preferred embodiment of FIG. 3, in which the embodiment further includes a rod 402 for rolling the shield 102 and a storage canister 404 for storing and transporting the shield 102 in a rolled configuration. The rod 402 includes an elongated slot 406 into which at least one end of the shield 102 can be inserted, so as to facilitate rolling of the shield 102. The shield 102 can then be stored within the storage canister 404, which can be closed with a lid 410.

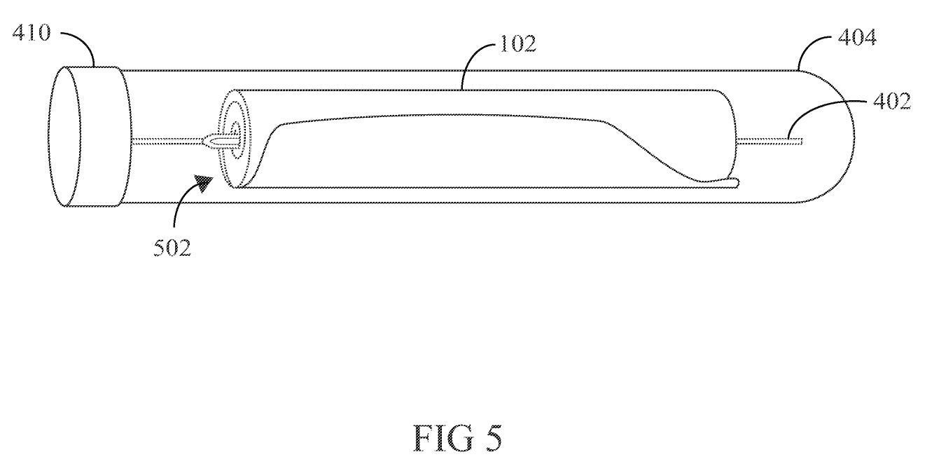

FIG. 5 is a perspective view of the light attenuating shield 102 of FIG. 4 rolled 502 around the rod 402 and inserted into the storage canister 404 so as to facilitate transportation and storage of the shield 102. The light attenuating shield 102 can be easily removed from the visor 104, rolled, and stored when not in use.

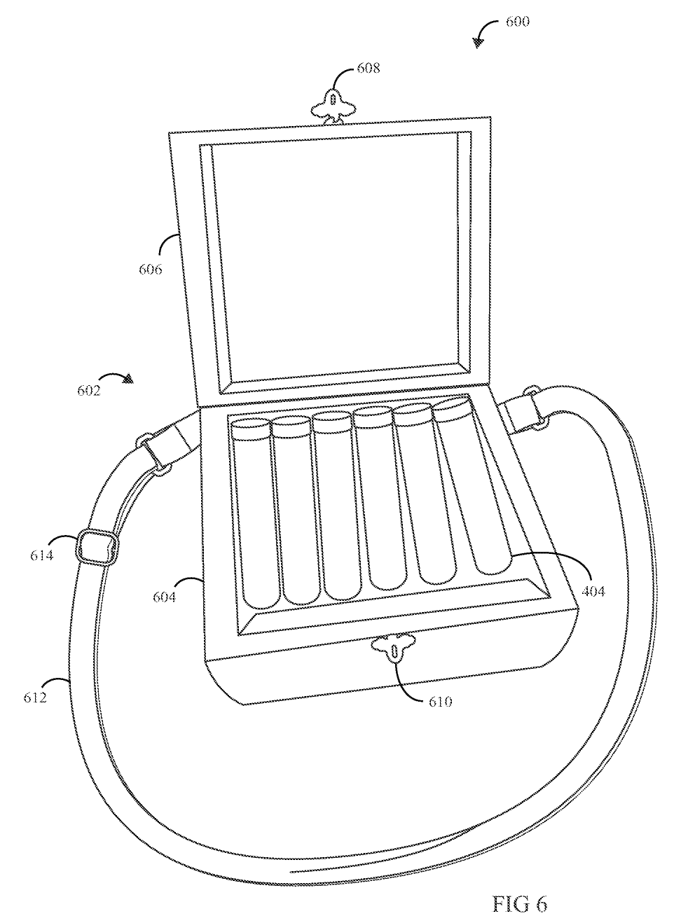

FIG. 6 is a perspective view of a preferred embodiment 600 that includes a plurality of light attenuating shields 102 rolled around rods 402 and contained in storage canisters 404. A storage box 602 is also included for convenient transportation and storage of the shields 102. In preferred embodiments, the plurality of shields 102 includes different types, arrangements, and degrees of tinting and/or polarization, so that the shields 102 can be carried and used by a motorcycle rider 110 as needed. In some embodiments, the shields 102 can be overlaid so as to optimize the light attenuation under various conditions.

The box 602 in FIG. 6 includes a lower tray 604 and a lid 606 that can be secured by a latch 608, 610. Each storage canister 404 has at least one light attenuating shield 102 stored therein. The embodiment of FIG. 6 includes a strap 612 having a length adjusting means 614 to facilitate carrying of the box 602 by a rider 110 while riding the motorcycle 208. In similar embodiments, the box 602 can be carried within a storage compartment of the motorcycle 114, or it can be attached to the motorcycle 114.

In certain embodiments the light attenuating shields 102 are disposable. In some of these embodiments, the shields 102 are either stacked within a dispensing package or contained in single-use canisters 404 that are discarded after use of the contained shield 102.

Other modifications and implementations will occur to those skilled in the art without departing from the spirit and the scope of the invention as claimed. Accordingly, the above description is not intended to limit the invention except as indicated in the following claims.

What is claimed is:

1. A light attenuating shield attachable to a motorcycle helmet visor, the light attenuating shield comprising:
a flexible sheet of polymeric film,
the flexible sheet being shaped so as to conform to a portion of an inner surface of a motorcycle visor,
the flexible sheet being able to attenuate light passing through the flexible sheet, and
the flexible sheet being removably attachable to the portion of the inner surface of the motorcycle visor by static electric attraction.

2. The light attenuating shield of claim 1, wherein the flexible sheet is tinted.

3. The light attenuating shield of claim 1, wherein the flexible sheet attenuates light in a non-uniform manner across the sheet.

4. The light attenuating shield of claim 1, wherein the flexible sheet polarizes light passing therethrough.

5. The light attenuating shield of claim 4, wherein light passing through the sheet is polarized so as to provide maximum attenuation of reflected glare.

6. The light attenuating shield of claim 1, further comprising at least one additional flexible sheet of polymeric film.

7. The light attenuating shield of claim 6, wherein a light attenuation characteristic of a first flexible sheet of polymeric film is different from a light attenuation characteristic of a second flexible sheet of polymeric film.

8. The light attenuating shield of claim 6, wherein at least two flexible sheets of polymeric film can be overlaid and simultaneously attached to the inside of the helmet visor.

9. The light attenuating shield of claim 1, wherein the flexible sheet is disposable.

10. The light attenuating shield of claim 1, wherein the flexible sheet is sufficiently flexible to enable transportation and storage of the flexible sheet in a rolled configuration.

11. The light attenuating shield of claim 10, further comprising a storage canister within which the flexible sheet can be stored when the flexible sheet is in a rolled configuration.

12. The light attenuating shield of claim 10, further comprising a rod for rolling the flexible sheet into a rolled configuration.

13. The light attenuating shield of claim 12, wherein the rod includes an elongated slot into which an end of the flexible sheet can be inserted so as to facilitate rolling of the flexible sheet.
